(19) United States
(12) Patent Application Publication
(10) Pub. No.: US 2008/0014575 A1
(43) Pub. Date: Jan. 17, 2008

(54) Rapid Microfluidic Assay for Quantitative Measurement of Interactions Among One or More Analytes

(75) Inventor: Kjell E. Nelson, Seattle, WA (US)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(73) Assignee: University of Washington, Seattle, WA (US)

(21) Appl. No.: 11/574,191

(22) PCT Filed: Oct. 25, 2005
(86) PCT No.: PCT/US05/38556

§ 371 Date: Feb. 23, 2007

Related U.S. Application Data
(60) Provisional application No. 60/622,193, filed on Oct. 25, 2004.

(51) Int. Cl. C12Q 1/70; C12M 1/34; G01N 33/487; C12Q 1/06

(52) U.S. Cl. 435/5; 422/68.1; 435/287.9; 435/39; 436/2

(57) ABSTRACT

The invention provides microfluidic competitive immunoassay devices and assay methods for rapid, quantitative measurement of binding interactions between analytes and the quantitative determination of an amount (e.g., concentration) of the analyte in an unknown sample.

### CROSS-REFERENCE TO RELATED APPLICATIONS

This application claims the benefit of U.S. provisional patent application No. 60/622,193, filed Oct. 25, 2004, the entire contents of which are incorporated herein by reference. Throughout this application, various patents and publications are referenced. The disclosures of these patents and publications are incorporated herein by reference to more fully describe the state of the art.

### STATEMENT AS TO RIGHTS TO INVENTIONS MADE UNDER FEDERALLY SPONSORED RESEARCH AND DEVELOPMENT

Aspects of this research were conducted with funding provided by the National Institute of Dental and Craniofacial Research under Grant No. 5U01 DE014971-03. The U.S. Government may have certain rights in the invention.

### BACKGROUND OF THE INVENTION

The present invention relates generally to a microfluidic competitive assay device and assay method. More specifically, the present invention uses an imaging assembly, such as surface plasmon resonance imaging to measure a rate at which analytes bind to a binding partner immobilized on a sensing surface of the device.

Most conventional competitive assays, such as, e.g., immunoassays, are carried out in an ELISA (Enzyme-Linked Immunosorbent Assay) format in which the presence and quantity of an analyte is determined by first immobilizing an antibody (or analyte) to a surface, exposing the treated surface to the unknown sample, rinsing off unbound molecules, then probing the surface with a second antibody conjugated to an enzyme or a fluorescent label which is used to generate the signal. Typically, these steps are performed in a plastic dish, though other formats are also used. Each step generally requires an incubation time between 30 minutes to an hour, meaning the time to assay a sample can be on the order of several hours. Quantitative data is obtained by comparing the results generated in a sample well (or sets of replicate wells) to a calibration set containing a number of other sets of wells, such as triplicates of five different concentrations (e.g., 15 wells). Creating this calibration series adds additional reagent cost and labor to the quantitative ELISA format, but is necessary to control for variations in assay time, reagent activity, and temperature.

The methods and devices of the present invention provide for these control conditions by generating a range of concentrations of a reference solution by virtue of diffusive mass transport during the experiment, eliminating the labor required and dramatically reducing the amount of reagent needed.

### SUMMARY OF THE INVENTION

The present invention provides microfluidic competitive assay devices and assay methods for rapid, quantitative measurement of interactions between an analyte and its binding partner that is immobilized on a sensing surface of the microfluidic assay device.

A concentration of an analyte in an unknown sample is determined by measuring a rate of binding of the analyte (e.g., an antibody) to a functionalized sensing surface of the microfluidic device in the presence of the analyte and comparing the rate of binding to a rate of binding observed in the presence of a reference solution containing a known concentration of a competitor. This comparison will provide information regarding the unknown concentration of the analyte in the sample. This comparison is typically though not necessarily done simultaneously with the measurement of the sample.

The methods of the present invention provide an improvement over conventional competitive immunoassays because quantitative determinations of multiple analytes in a single small fluid sample (e.g., <0.1 mL) can be made rapidly and simultaneously with a reference solution. Additionally, the methods of the present invention do not require the addition of a labeled component to the sample prior to measurement. Moreover, by selecting particular fluidic geometries of the microfluidic competitive immunoassay device, the measurements can include real-time comparisons to reference solutions to control for variations in temperature, detector response, and other manufacturing uncertainties. These controls can be done simultaneously with the sample measurement and therefore do not increase the time required to conduct the assay.

The microfluidic devices of the present invention are typically in the form of an inexpensive, disposable microfluidic cartridge (a “lab on a chip”) and associated automated imaging and processing equipment. Such devices are exceptionally well suited for running rapid, multiple analyte assays, such as immunoassays. Thus, the devices of the present invention establish a solid basis for reliable point-of-care diagnostics by relatively untrained personnel, although it could be used in larger formats in clinical laboratory settings as well.

The analytes that may be analyzed by the present invention include small molecules, antibody/antigen conjugates, nucleic acids, nucleic acid/protein interactions, or other protein/protein interactions, or larger particles (such as viruses or bacteria). Depending on the format of the assay implemented, one or more analytes in a single sample fluid volume can be measured simultaneously. Typical analytes for detection and measurement via the invention include antibodies, antigens, nucleic acids, and proteins.

The competitive assay devices of the present invention operate similar to other competitive immunoassay devices, but do not require an enzyme-linked or fluorescently tagged secondary antibody, nor do they require the addition of a labeled competitor species or analog. Instead, the present assay devices use an imaging assembly, such as surface plasmon resonance imaging (SPRI) assembly, that provides for measuring a rate at which antibody molecules bind to specific antigens immobilized on a sensing surface, or vice-versa. The presence of free (i.e., solution phase) competitors reduce the rate of antibody adsorption to the antigens on the sensing surface by binding to their antigen binding sites.

These and other aspects of the invention will be further evident from the attached drawings and description of the invention.

### BRIEF DESCRIPTION OF THE DRAWINGS

FIG. 1 schematically illustrates a simplified system encompassed by the present invention.

FIG. 2 is a plan view of one embodiment of a microfluidic competitive immunoassay device encompassed by the present invention.

FIG. 2A is a simplified cross sectional view of a competitive immunoassay encompassed by the present invention.

FIG. 2B is an exploded view of one embodiment of a microfluidic competitive immunoassay encompassed by the present invention.

FIG. 3 illustrates a geometry of a microfluidic channel of the microfluidic competitive immunoassay device of the present invention that may be used to establish a concentration gradient of diffusing analytes and its binding partner in bulk phase.

FIG. 4 illustrates a concentration profile of immunoassay reagents at various positions in the microfluidic channel. The data are based on the diffusion coefficients of an IgG antibody, a small molecule (such as biotin) and the product of the two concentrations to suggest potential concentration profiles.

FIG. 5 illustrates concentration profiles of uncomplexed antibodies at various positions in the microfluidic channel. The rate of adsorption of the uncomplexed antibody is proportional to its concentration at a given channel position. Therefore, the rate of adsorption at position 220, where the concentration of the antibody/antigen complex is non-zero, will be lower than at other positions (e.g., from 400-600).

FIG. 6A depicts three cross sectional slices in the microfluidic channel, which illustrate different concentration gradients of a competitor molecule.

FIG. 6B illustrates an SPRI image created from signals from the SPRI sensing surface of FIG. 6A. The SPRI image shows the position dependent variation in the rate of antibody accumulation to the SPRI sensing surface caused by the diffusing competitor.

FIG. 6C illustrates the patterns, gradients and profiles observed in various regions of a microfluidic channel (shown in the center). The lower left inset shows the SPRI pattern in relation to the antibody stream width in the surface binding sensing region. The upper left inset depicts the relative binding of antibody, free antigen and surface-bound antigen across the analyte concentration gradient in this sensing region. The three insets to the right illustrate the concentration profile as a function of channel position at three different points along the interdiffusion zone.

FIGS. 7A and 7B illustrate an example of a protein pattern that may be used for an immunoassay device and method of the present invention for the simultaneous detection of multiple analytes in a single fluid sample.

FIG. 8 illustrates SPRI results that demonstrate position dependent SPRI response due to varying concentrations of competitors in a parallel immunoassay.

FIG. 9 schematically illustrates an example of an experimental protocol encompassed by the present invention.

FIG. 10 is a representative plot of antibody distributions around a fluid stream interface for six different channel positions. The distribution is calculated based on a 1-dimensional Fickian diffusion model using a 150 KDa molecule (IgG) and does not take into account the presence of an analyte or complex.

FIG. 11 is a plot of representative distributions of a low-molecular weight compound around the fluid stream interface for six different channel positions. The distribution is based on a 1-dimensional diffusion model and (˜250 Da) molecule (biotin) and does not take into account the presence of antibodies or antibody/analyte complex.

FIG. 12 is a plot of representative distributions of antibody/antigen compound around the fluid stream interface for six different channel positions. The distributions are shown to suggest possible concentration profiles based on the product of the data shown in FIGS. 10 and 11 and are not intended to accurately reflect any specific result of the assay method.

FIG. 13 is a plot of representative distribution of uncomplexed antibody around the fluid stream interface for six different channel positions. This distribution is calculated based on difference between antibody and complex distribution at channel positions indicated in legend. Note that the rate of antibody binding to immobilized antigen is proportional to the concentration at each transverse channel position (i.e., rate of binding is highest on right side of channel and drops off rapidly near fluid stream interface).

### DETAILED DESCRIPTION OF THE INVENTION

The present invention provides methods, competitive assay devices, kits, and systems that are configured to, determine an unknown concentration of one or more analytes in a fluid sample.

The assay of the present invention, typically an immunoassay, can be adapted to work downstream of microfluidic sample pre-conditioning methods to enable detection of small molecules in a variety of clinical samples (e.g., saliva, serum, whole blood, CSF, urine, stool, pulmonary fluid, etc.), making it well suited for integration into “lab on a chip” microfluidic systems and allowing for a high degree of automation. Taken together with the rapid time to obtain a result, the present invention is much better suited to point-of-care diagnostic testing by relatively untrained personnel than standard immunoassay formats.

While the assays of the present invention are described herein as an immunoassay, the present invention may also be applied to detect and quantitatively measure interactions among nucleic acids, proteins, peptides, polypeptides, hormones, small molecule binding partners, etc., and is thus much more versatile than a standard competitive immunoassay, which is used only to measure interactions between an antibody and its conjugate antigen.

One advantage to the present invention is that, in contrast to standard ELISAs, in which each analyte must be measured individually in a given fluid sample, multiple analytes can be measured in parallel within the same assay and device. A farther advantage the present invention provides over standard ELISAs is that the volume of reagents required is quite small (˜75 uL), whereas standard ELISAs often require on the order of at least several milliliters (or more). The assay described herein has additional advantage of producing a quantitative result based on the rate of a process (rather than an endpoint). Therefore, it can generate a result, complete with internal controls and references, for all analytes within 15 minutes, and preferably less than 5 minutes following sample introduction.

The present invention typically uses an external imaging assembly, such as an SPRI assembly rather than color changes or the presence of fluorescently labeled secondary antibodies, enabling the label free detection of only those species of antibody that bind to the sensing surface. This eliminates the need for complex fluorescence resonance energy transfer (FRET) based reagents designed to discriminate between bound and unbound antibodies, although these detection methods could be implemented if proven to be suitable for a particular application. Moreover, the digital images generated by SPR can be processed automatically to provide an untrained user with valid and reliable quantitative data.

Although this method may appear similar in some respects to methods based on a similar microfluidic format, such as the T-sensor or the Diffusion Immunoassay (described in U.S. Pat. Nos. 5,972,710 and 6,541,213 to Weigl and U.S. Pat. No. 5,716,852 to Yager, the complete disclosures of which are incorporated herein by reference), the present invention differs from them in at least the following respects: 1) detection occurs following binding of an uncomplexed species to a surface, 2) determination of the concentration of analyte occurs as a result of competition between the sample and a species immobilized on the sensing surface, 3) the assay does not rely on the establishment of a differential rate of diffusion between the antigen and its complexation with antibody, 4) multiple analytes can be detected simultaneously within the same stream, for example, by patterning the sensing surface with different antigens, 5) the assay does not require the addition of a labeled species (e.g., a competitor species conjugated to a fluorescent dye or an enzyme), 6) the assay does not require that one of the fluid streams contain an indicator, 7) the devices used in this method are not reusable.

Detection of analyte using the present devices and methods is typically on the order of 10 nM analyte, with a dynamic range on the order of three orders of magnitude. The dynamic range of the assay can be tailored to suit a particular application by changing the concentration of the species used in the assay stream, in this case concentration of the antibody in the center stream. Combined with SPR image enhancement strategies, detection of analyte into the upper pM range may be achieved, with a concomitant increase in dynamic range.

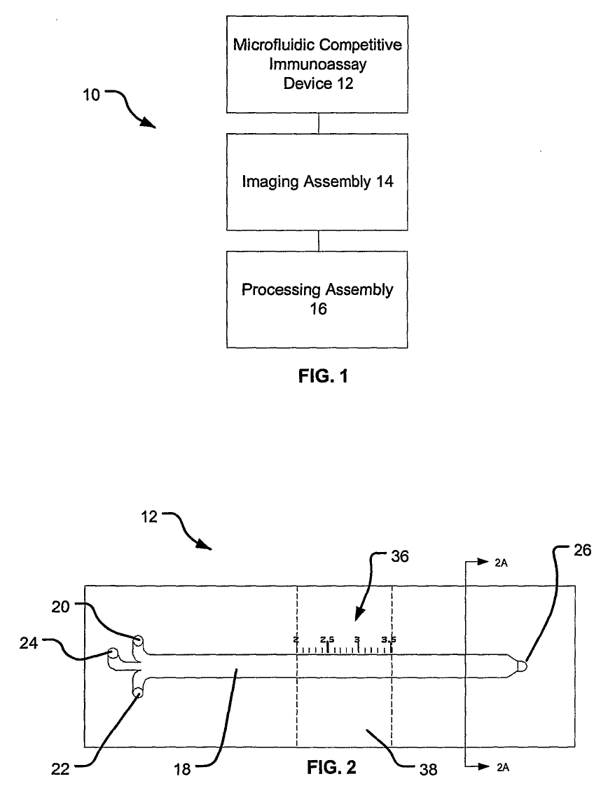

FIG. 1 schematically illustrates a simplified system of the present invention. The system 10 of the present invention comprises a microfluidic competitive immunoassay device 12 that is adapted to receive a plurality of fluid streams—including the fluid sample having an unknown concentration of analyte. An automated, external imaging assembly 14 is optically coupled to a sensing surface of the competitive immunoassay device 12 to measure a rate of binding of the analyte to the sensing surface. Information about the rate of binding and the concentration profile of the analyte will be in a signal generated by the imaging assembly 14 to help determine a concentration of the analyte in the unknown fluid sample. Imaging assembly 14 may be electronically coupled to a processing assembly 16 to process the signals from the imaging assembly 14 to generate the desired data and outputs. The systems 10 of the present invention typically are able to determine a concentration of one or more analytes in a small fluid volume (e.g., <0.1 mL) in a short period of time (e.g., less than about 15 minutes).

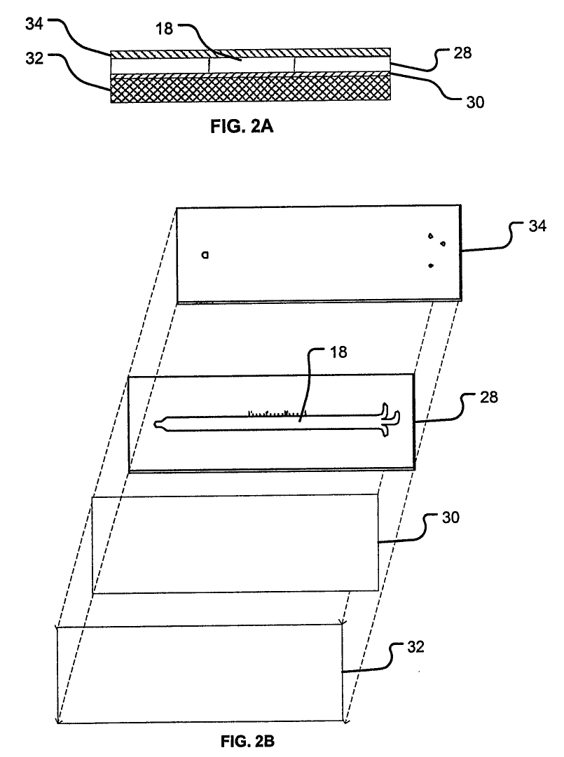

FIG. 2 illustrates one embodiment of a microfluidic competitive immunoassay device 12 encompassed by the present invention. The microfluidic competitive immunoassay device 12 is typically in the form of a disposable microfluidic cartridge (e.g., “lab on a chip”). The microfluidic device comprises a microfluidic channel 18 that has a plurality of inlets for receiving different fluid flows. The microfluidic channel 18 has at least a first inlet 20 and a second inlet 22, but may optionally comprise additional inlets. As shown in the embodiment of FIG. 2, there is an optional, third inlet 24. As can be appreciated by those of ordinary skill in the art, while three inlets 20, 22, 24 are illustrated, the microfluidic competitive immunoassay devices 12 of the present invention may comprises any number of inlets and the present invention is not limited to the illustrated number of inlets. The fluid flows from inlets 20, 22 (and 24) flow down microfluidic channel 18 and exit the microfluidic channel 18 through an outlet 26.

In one embodiment as shown in FIGS. 2 to 2B, the microfluidic channel 18 is formed in Mylar® sheet 28 that has a thickness between about 50 μm to about 100 μm. The Mylar® sheet 28 may be coated on both sides with 25 μm of adhesive (not shown). As shown in FIG. 2B, the Mylar® sheet 28 may be cut to create the microfluidic channel 18. The Mylar® sheet 28 may be fixed directly to a gold-coating 30 on a microscope slide 32. The gold coating 30 forms a sensing surface for the SPRI assembly 14. The gold coating may have different thickness but is typically about 45 nm thick. Moreover, other metals (e.g., silver or aluminum) may be used if deposited in appropriate thicknesses known to those with ordinary skill in the art, and dielectric coatings may be deposited on top of the metal films to suit a particular application. A second sheet 34, of either Mylar® or Rohaglas® that has a thickness of about 100 μm or thicker, may be cut to create the inlets 20, 22, 24 and outlet 26, and thereafter affixed to the first Mylar sheet 28 to form a cap and complete the microfluidic competitive immunoassay device 12.

The microfluidic competitive immunoassay device 12 has one surface of the microfluidic channel 18 that is adapted to be a sensing surface 38. Sensing surface 38 is coated with a binding partner to the analyte. When the analyte is an antibody, the sensing surface is patterned with an antigen, such that the antibody can bind to the sensing surface as well as to the bulk phase competitors. Coating the sensing surface can be accomplished by any number of available methods (including, but not limited to, passive adsorption or conjugation to a reactive chemical groups present or deposited on the surface). Thus, in such embodiments, the competition will be between the bulk phase, competitor antigens and surface bound antigens. Optionally, the microfluidic channel may comprises fiducial markings 36 around the sensing surface 38 of the SPRI assembly 14 to aid in device and assay characterization.

The portion of the channel upstream of the sensing region is typically treated with a coating designed to reduce or prevent adsorption of molecules from the fluid stream to the channel walls. This can be done with a number of different methods, including but not limited to passivating the surface with BSA, casein, or poly(ethylene glycol) (PEG).

For ease of reference, a fluid flow manifold (e.g., pumps, tubings, fittings, etc.) and the SPRI imaging assembly 14 are not shown, but a person of ordinary skill in the art will appreciate that such elements are coupled to the device 12 shown in FIGS. 2 to 2B.

The competitive immunoassays 12 of the present invention are based on a reduction of binding of target analytes (e.g. antibodies) to an immobilized binding partner positioned on a sensing surface of the microfluidic channel 18 within the sensing region 3 8, due to the binding of a competitor molecule to the analyte while the analyte and competitor are both still in the bulk solution phase. For example, if the analyte is an antibody, binding of the competitor to the antigen recognition site of the antibody prevents specific binding of the antibody to the surface-bound antigens (or reduces the probability of binding in the case of a single competitor molecule bound to a divalent antibody.)

Conversely, the methods and devices of the present invention can also be used in situations wherein it is more convenient for the antibody to be bound to the sensing surface 38 and competition for the antibody binding sites occurs between the bulk phase analyte and the competitor analyte (which may optionally be labeled). Thus, while the discussion focuses on binding an antigen to the sensing region, the present invention further encompasses methods which bind the antibody to the sensing surface 38.

The methods of the present invention rely upon the rate of analyte binding to the sensing surface 38 within the microfluidic channel 18. The rate of binding is inversely proportional to the concentration of competitor present in the bulk phase and the relative concentrations of analyte and competitor. In other words, the higher the concentration of competitor relative to the concentration of the analyte, the greater the proportion of analyte-competitor complexes compared to free analyte, and the slower the rate of accumulation of analyte (antibody or antigen) to the sensing surface 38.

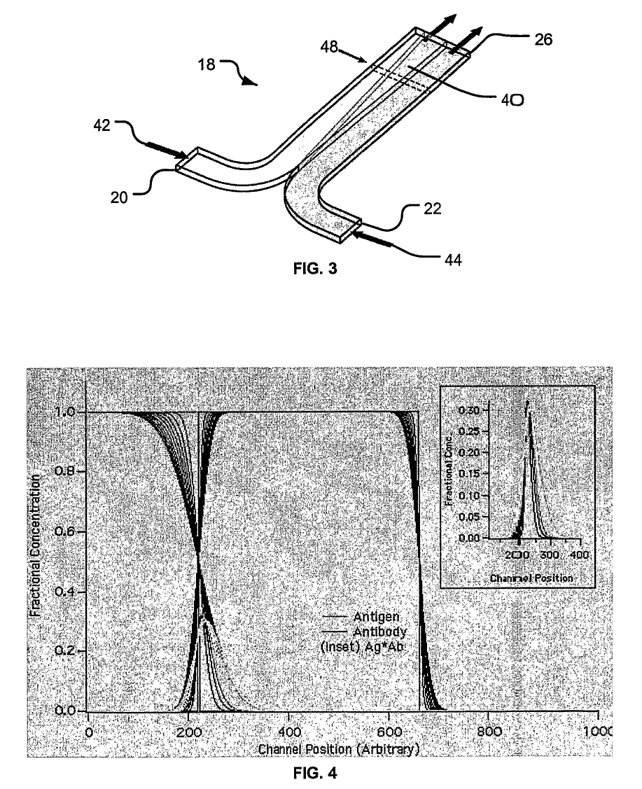

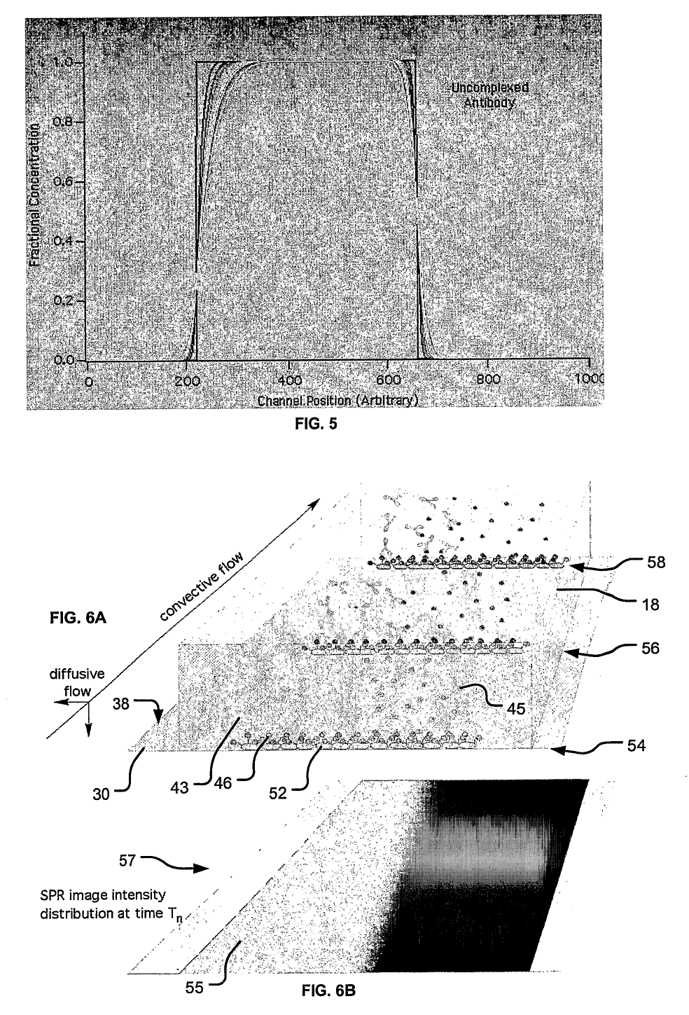

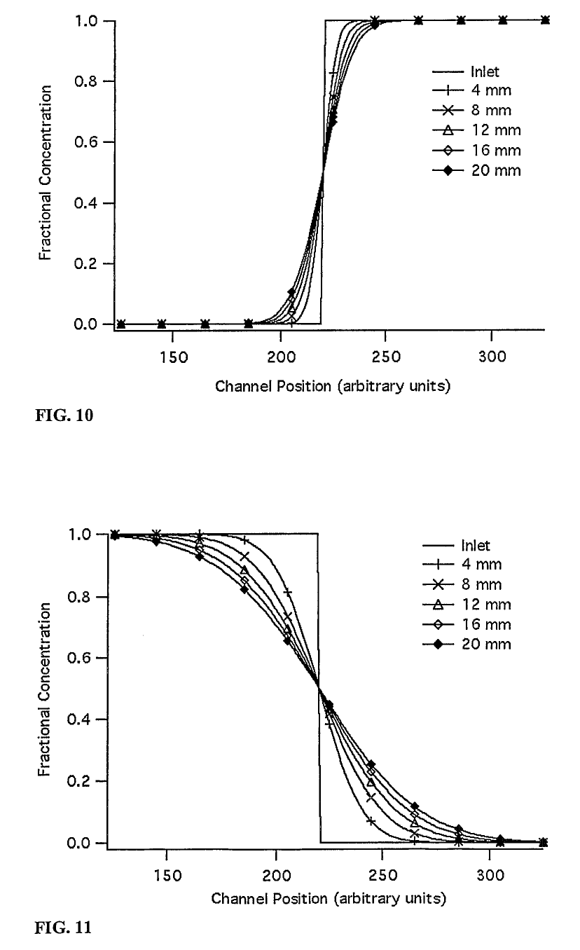

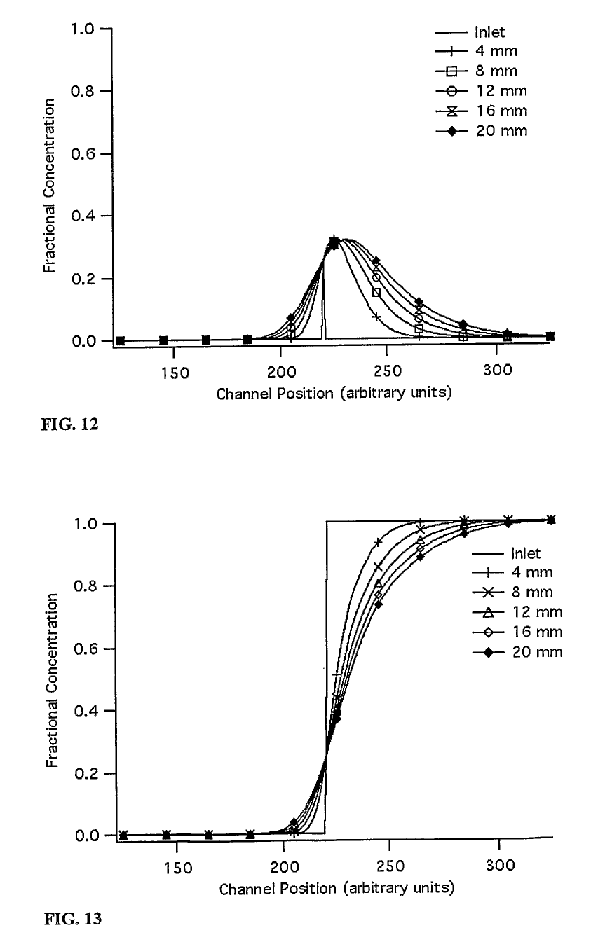

The assay methods of the present invention develop a concentration profile 40 of competing species perpendicular to a bulk flow in the microfluidic channel 18. One implementation of the method of the present invention is shown in FIG. 3 and makes use of different concentration gradients along the microfluidic channel 18 to carry out competitive immunoassays by flowing a first stream 42 of buffer containing an analyte and an adjacent second stream 44 of buffer containing an unknown concentration of the competitor. As the two streams 42, 44 inter-diffuse down the length of the microfluidic channel 18, the proportion of analytes (e.g., antibodies) in the first stream 42 bound to the competitor in the second stream 44 depends on the local concentration of competitor (FIG. 4; FIG. 10-13). As a result of the establishment of different concentration gradients at different locations down the microfluidic channel 18, the analytes in the first stream 42 will encounter different concentrations of the competitor based on a position in the microfluidic channel 18. Consequently, a concentration profile 40 of unbound analytes will be generated throughout the microfluidic channel that is stable over time at a particular location (FIG. 5). Moreover, the specific concentration profile developed will depend on the concentration of the analyte in the sample.

In embodiments where the analyte is an antibody and the competitors and surface bound binding partner are antigens, the unbound antibodies are capable of binding to the surface-bound antigens 46 along the sensing surface 38. Again, the rate of binding of the antibody to the surface-bound antigen 46 is proportional to the concentration of unbound antibody, which varies across the width of the microfluidic channel 18 and depends on the given position downstream of the fluid inlets 20, 22. A simple depiction of how such a concentration profile can be established in the microfluidic channel 18 is shown in FIG. 3. While the concentration gradient is typically along a width of the microfluidic channel 18 (and substantially orthogonal to the fluid flow), the direction of the concentration gradient 40 of the competing species is not necessarily along the width of the channel as shown in FIG. 3

In the example illustrated in FIG. 3, the competitor, which is a rapidly diffusing binding partner antigen, is carried in the second fluid stream 44 and the antibody which is a slowly diffusing species, is carried in the first fluid stream 42. As the two streams 42, 44 flow down the microfluidic channel 18 adjacent to each other, the competitor diffuses across the interface between the two streams so as to establish the concentration profile 40. The arrow 48 points to a specific position downstream of the fluid inlets where the concentration profile at this (and all other) position in the fluid stream is stable over time.

As a result of the laminar flow that occurs in a low Reynolds number flow that characterize fluid dynamics in a microfluidic channel 18, the concentration profile 40 generated is predictable and reliable as a result of diffusion-based mass transport across the interface between the adjacent fluid flows 42, 44 containing different concentrations of solute. The longer the two fluids 42, 44 remain in contact, the shallower the concentration gradient 40 will be between them. Since the concentration gradient 40 is established by allowing the two fluids to flow adjacent to each other, different concentration profiles are created at different positions down the microfluidic channel 18. Given that the flow rate is constant and does not change over the course of an immunoassay, the concentration profiles are stable over time at any given position in the microfluidic channel 18.

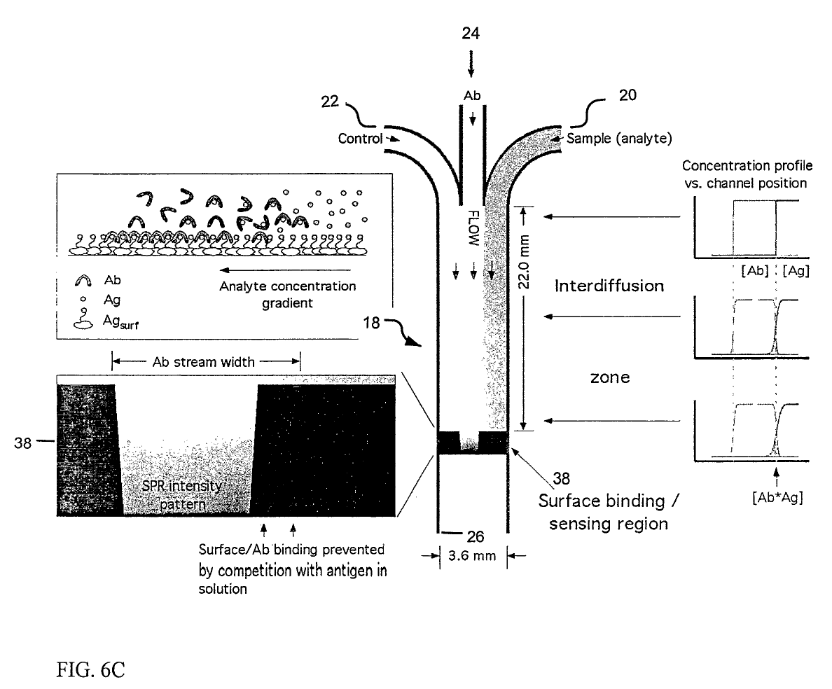

FIGS. 6A-6B schematically illustrate three different concentration profiles along three different longitudinal positions within the microfluidic channel 18. In the illustrated embodiment, the analyte is an antibody 43 and the competitor and the surface bound binding partner are antigens. The rate of antibody 43 binding to the surface-bound antigen 46 is measured using surface plasmon resonance imaging (SPRI) (FIG. 6B), although other conventional types of detection formats are possible. As is known in the art, SPRI is a spectroscopic technique that is sensitive to changes in the dielectric properties of the medium immediately adjacent (<0.5 um) to a metal surface (e.g., gold coating 30). An SPR signal is changed when the antibody 43 binds to the immobilized antigen 46 on sensing surface 38.

In the illustrated embodiment, the entire microfluidic channel sensing surface 38 in this case is coated with an adhesive, such as a bovine serum albumin (BSA)-derived conjugate 52 that immobilizes the antigen 46 to the sensing surface 38. In the first cross-sectional position 54 in the microfluidic channel 18, free antigen 45 in bulk phase is present in the fluid stream on the right (e.g., second stream 44 in FIG. 3). The antibody 42 is in the stream on the left (e.g., first stream 42 in FIG. 3). Antibody 43 is able to bind to the antigen 46 immobilized on the surface 38 across the channel 18 generating a bright region 55 in the SPR image 57 (FIG. 6B). Further downstream in the second cross-sectional position 56, where the competitor antigen 45 stream has had time to diffuse into the antibody 43 stream and bind with the antibody 43, the concentration of free, unbound antibodies is lower. Hence, the amount of antibody 43 accumulation near the fluid interface downstream is less than it is upstream. Finally, in the third cross-sectional position 58, downstream of the second position 56, the antigen 45 stream diffuses even deeper into the antibody 43 stream and further reduces the amount of free antibodies in the antibody stream. As shown in FIG. 6B, the bright region 55 reduces moving downstream as the competitor, antigen stream diffuses into the antibody stream and reduces the binding of the antibody 43 to the surface immobilized antigens 46.

Since the present invention does not require a label, SPRI eliminates the need for indirect detection schemes required for many conventional immunoassays. For example, some conventional methods use secondary antibodies labeled with either fluorescent tags or enzymes capable of generating a colored product from a colorless substrate. Eliminating the need for such a tagged antibody reduces the labor, time, and cost required to carry out the assay.

In some configurations, the microfluidic channel 18 may include three or four (or more) adjacent fluid streams (FIG. 6C). In such embodiments, a first fluid stream may contain a competitor reference solution that has a known concentration of the analyte. A second fluid stream carries the unknown sample and flows in parallel down the microfluidic channel 18. A third fluid stream may flow between the first fluid stream and the second fluid stream. The third fluid stream contains an antibody (or antigen) that can bind with the analyte in the first and third fluid streams. Such an embodiment enables real-time, on-chip referencing and controls without increasing the time required to conduct the assay, and only slightly increases the amount of reagent required. In use, the three fluid streams simultaneously enter the microfluidic channel 18 from each of the three inlets 20, 22, 24. The fluids are injected at a constant and equal flow rate (e.g., approximately 22 nL/sec) The three fluid streams flow down the microfluidic channel and exit through outlet port 26 and pass over the sensing surface 38, as described above.

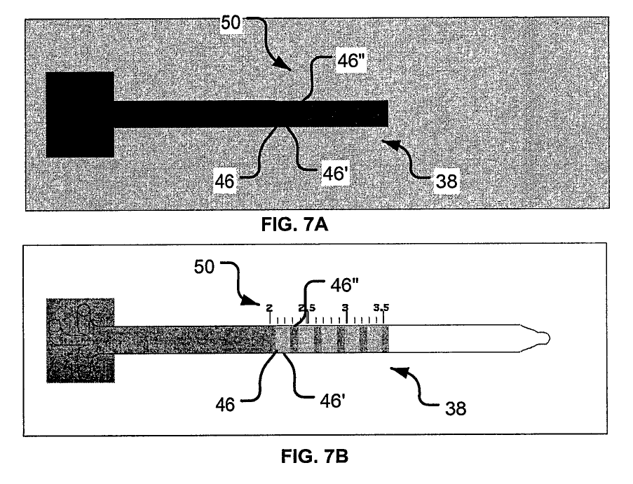

The microfluidic competitive immunoassay device 12 of the present invention may optionally be modified to conduct multiple simultaneous assays. The simultaneous assays may be carried out by patterning a number of different surface-bound antigens 46, 46′ (or antibodies) within the sensing surface 38 (FIGS. 7A and 7B). In such an embodiment, any number of non-cross-reactive antibodies (or antigens) are dissolved in solution flowing in the center inlet 24 and the competitors are similarly mixed together in the first fluid stream and are flowed through first inlet 20. Parallel detection occurs when a given antibody (or antigen) traverses the region of the sensing surface 38 that has been modified with its binding partner, where the surface has multiple binding partners spatially addressed within the sensing surface 38 (FIGS. 7A and 7B). Diffusion and binding between each different antibody (or antigens) and their competitors occur within the same flow streams. Therefore, the number and types of simultaneous assays possible with this format is limited only by the ability to pattern antigens 46, 46′ (or antibodies) in the sensing surface 38, the resolution of the imaging assembly 14, and the availabilities of monoclonal antibodies specific for the analyte of interest. The time required to conduct the multiple analyte detection in this case does not significantly differ from the time required to carry out a single assay.

One example of patterning different antigens includes a pattern of BSA 46, BSA-cortisol conjugate 46′, and/or BSA-estriol conjugate 46″. A sensing surface 38 patterned in this way results in a sensing surface 38 that allows for inter-diffusion between the adjacent streams upstream of the sensing region without interacting with the sensing surface 38, then allowing for binding of the uncomplexed antibody to specific binding partners within a given patterned region.

### EXAMPLE I

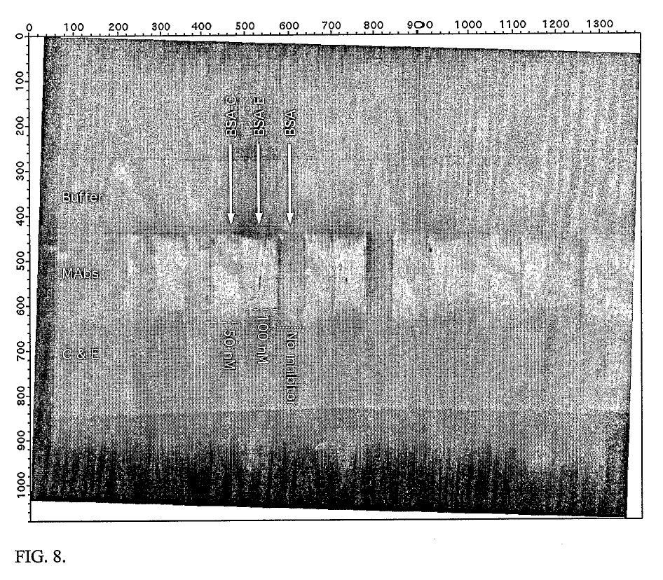

As shown in FIG. 8, the immunoassay method of the present invention has been demonstrated experimentally by measuring two analytes in parallel at several regions in a single microfluidic channel. In such experiments, three streams are flowing parallel from the inlets to the outlet. As shown in FIG. 8, flow is from left to right. The first fluid stream comprises a buffer only and is included as a negative control. The second, middle fluid stream comprises a mixture of anti-cortisol and anti-estriol monoclonal antibodies (100 nM each) (shown as “MAbs”). The third fluid stream comprises a buffer with cortisol (50 nM) and estriol (100 nM) (shown as “C & E”). The sensing surface had been patterned with similar surface densities of BSA, BSA-cortisol conjugate (“BSA-C”), and BSA-estriot conjugate (“BSA-E”). The BSA/BSA conjugate triple pattern was repeated five times from left to right. The labels in FIG. 8 are positioned at the second repeated triplet. The gold coating was treated with BSA to the left of pixel column ˜240 (to prevent non-specific antibody binding) and was untreated to the right of pixel column ˜1250 (allowing non-specific antibody binding). The narrower area of antibody binding to the sensing surface within the BSA-estriol conjugate regions (as indicated by the bright regions in the image and demarked by dashed lines) resulted from the higher concentration of estriol in the competitor stream relative to the cortisol concentration. Time to obtain this result is typically less than 15 minutes, and preferably about 5 minutes.

In the present invention, the dynamic range of the assay can be varied by changing the concentration of antibody in solution. The higher the concentration of antibody, the more competitor will be required to effectively establish a concentration gradient of unbound antibody and thus a detectable variation of the rate of change in the SPR image.

### EXAMPLE II

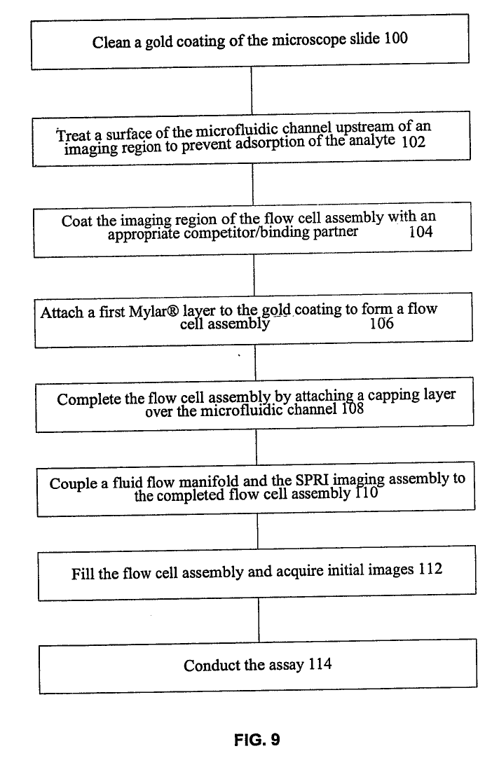

One example of a non-limiting protocol of the present invention will now be described. A simplified flow chart illustrating the experimental protocol is provided in FIG. 9. At step 100, the gold coating of the microscope slide is cleaned. However, if the glass slides have been freshly evaporated (within the previous 60 minutes), the cleaning steps of the gold coating can be omitted. The gold coating may be cleaned in a hot base/peroxide wash In such a method, in a clean, flat-bottom glass dish, hydrogen peroxide, ammonium hydroxide, and ddH₂O are mixed in a 1:1:5 volumetric ratio (e.g., 10 mL H₂0₂, 10 mL NH₄OH, 50 mL ddH₂O). The solution is heated to 65-75° C. and covered with a watch glass to minimize evaporative loss. The gold coated glass slide is immersed in the heated solution and soaked for approximately 10 minutes. The slide is removed and rinsed first with ddH₂O then absolute ethanol. Finally, the slide is blow dried under a dry N₂ stream. Other methods of cleaning the gold film known to those with ordinary skill in the art may also be used.

At step 102, the surface of the microfluidic channel may then be treated upstream of a sensing surface so as to reduce, and preferably prevent the adsorption of the solution phase analytes to the surface upstream of the sensing surface. For example, the gold coating upstream of the imaging region is treated with a bovine serum albumin (BSA) in a phosphate buffer (PB). In such an embodiment, the flow cell assembly is placed into an empty 50 mL centrifuge tube. The user then visually determines the amount of solution required that will fill the centrifuge tube so, that the solution just reaches the level of the sensing surface on the microscope glass slide (as indicated by fiducial marks on the Mylar® layer). Typically, the amount needed is about 25 mL. The flow cell assembly is removed from the centrifuge tube and the centrifuge tube is placed in a rack to hold it vertical. An appropriate amount of PB containing 5 mg/mL BSA is added to the previously determined level. Care should be taken to avoid bubbles in the BSA solution. The flow cell assembly is placed into the tube with the inlet ports first, such that the level of PB/BSA wets the channel up to just inside the sensing surface that is defined by the fiducial marks. The flow cell assembly is incubated in the blocking solution at room temperature for at least 60 minutes, and preferably overnight. The slide may then be removed from the blocking solution and rinsed with water using a rinse bottle with the stream and waste directed toward the inlet port side of the slide (e.g., away from the sensing surface). The blocking and rinse solution should be prevented from contacting the microfluidic channel within the sensing surface. Finally, the flow cell assembly is blown dry with N₂. Other methods for patterning the upstream region may be used.

As can be appreciated, other known methods of preventing the adsorption of proteins or analytes to a gold coating may be used. For example, the gold coating upstream of the sensing surface may be coated with ethylene-oxide terminated SAM prior to assembly, if desired.

At step 104, the sensing surface of the flow cell assembly is coated with the appropriate competitor for the intended assay. In one method, approximately 50 μL of 5 mg/mL BSA-conjugated competitor (BSA-C) is placed onto the bare gold coating of the microfluidic channel within the sensing surface and the droplets are spread across the sensing surface of the microfluidic channel with a pipet tip. The flow cell assembly is allowed to sit undisturbed, face up, covered, for 60 minutes. Thereafter, the remaining coating solution is rinsed off of the microfluidic channel with a wash solution. The wash solution should be directed to drain away from the inlets and imaging region and toward the outlet, so as to not contact the area upstream of the imaging region. Finally, the flow cell assembly is dried with N₂.

A parallel-throughput immunoassay can be carried out if the following series of steps are substituted with currently available protein printing technology used to create an array of transverse strips 1 mm wide of different competitors immobilized in the sensing region. When used in this format, the antibody stream contains not one, but a mixture of non-cross reacting antibodies, one for each of the different conjugates immobilized in the protein array. The BSA competitor used in this example is BSA-cortisol. The BSA conjugate used in this example to immobilize the competitor to the sensing surface can be replaced with, for example, an antibody or other molecule (such as a gene regulatory protein or nucleic acid) using any one of a number of bioconjugate chemical techniques, and the solution-phase molecule selected accordingly to complete the operation of the competition assay.

At step 106, a first Mylar® layer is attached to the gold coating. One of the protective layers from the Mylar® adhesive coating layer is removed. The edges of the Mylar® layer are aligned to the edges of the microscope slide. The Mylar® layer is pressed to adhere it to the gold-coated side of the microscope slide. The microfluidic channel is preferably already formed in the Mylar® layer prior to attaching the layer to the gold coating. The edges of the Mylar® layer may thereafter be pressed to the edges of the gold coating to ensure a good seal around the edges of the microfluidic channel. The combination of the gold coated microscope slide and the Mylar® layer is referred to herein as “flow cell assembly.”

At step 108, the flow cell assembly is completed. A protective backing is removed from the top of the Mylar® ACA sheet. The edges and ports formed in the capping layer (e.g., second Mylar® or Rohaglas® layer) is aligned with the flow cell assembly and pressed to secure the adhesive. Using a smooth, narrow tool such as the back end of a dental pick, the layers are pressed together so that the edges of the channel and around the ports gave good adhesion, channel acuity and to prevent potential leaks and cross-contamination between channels.

Once the microfluidic competitive immunoassay device is completed, fluid flow manifold and a SPR imaging assembly may be coupled to the capped flow cell assembly, step 110. For example, tubings and fittings that are coupled to three pumps that are capable of delivering less than 30.0 nL/sec are coupled to each of the inlets. A flow cell assembly holder with tubing ports and gaskets for leak-free attachment of the pump tubing are coupled to the flow cell assembly inlet ports. Sample loops and valves may be used to regulate the composition of the solutions connected to the inlet ports (e.g., a means to switch from buffer to sample solutions.)

The surface plasmon resonance imaging assembly with associated imaging optics (e.g., a CCD camera) and data acquisition and storage capability (e.g., the processing assembly 16) are positioned adjacent the flow cell assembly. A variety of different SPRI configurations are possible and acceptable given the capability of imaging a ˜1.5 cm length of the channel (e.g., the sensing surface) at 50 μm spatial resolution or better.)

At 112, once the input fluid flow manifold is coupled to tile inlet ports, the flow cell assembly is filled and initial images are acquired. In this step, the microfluidic device is filled with ddH₂O so as to ensure the removal of all bubbles. Thereafter, the pumps and tubing are filled and flushed with running buffer (e.g., 10 mM phosphate buffer (PB). The flow cell assembly is coupled to an SPPI prism using an index matching oil. The pump tubing is connected to the flow cell manifold, again to ensure that no bubbles are present. The light intensity and image integration time of the SPRI instrumentation is set to maximize the dynamic range of the image signal and the coupling wavelength (or angle) of the SPRI assembly is set such that the intensity of the image in the channel is near the SPRI minimum but also on the edge of the linear region of the slope of the SPRI spectrum. Transverse magnetic (TM) and transverse electric (TE) polarization images of the initial condition of the channel may then be acquired.

Finally, at 114, the assay is conducted. 100 [IL of each of the assay solutions (antibody, sample, and reference) is loaded into one of the three sample loops. The pump tubing is connected with the flow cell manifold and it is checked to ensure that no bubbles are present within the system, particularly upstream of the sensing surface. TM data acquisition (e.g., one image frame every 10 seconds) is begun and data representation benefits from normalization to the intial TM image (i.e., collect difference images to highlight the changes in SPR over time). Fluid flow is initiated, e.g., 29 nL sec⁻¹ channel⁻¹ for a rapidly diffusing species such as cortisol. Data acquisition is continued for approximately 10 minutes, which should be sufficient to observe the change from water to PB, followed by the accumulation of antibody on the sensing region. Data acquisition may be extended if lower antibody concentrations are used to lower the limit of detection or if longer channels are used for more slowly diffusing species. Finally, the slope and position of maximum signal of the interfaces is compared between the reference and antibody streams and the sample and antibody streams to determine the concentration in the sample.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for analyzing a sample stream for quantitative detection of one or more analytes, the method comprising:
providing a laminar flow microfluidic channel that comprises a first inlet, a second inlet and an outlet;
immobilizing a binding pair of the analyte(s) on a sensing surface of the laminar flow microfluidic channel;
delivering a first stream into the first inlet, the first stream comprising the one or more analytes;
delivering a second stream into the second inlet, the second stream comprising binding pair(s) to the one or more analyte(s) of the first stream, wherein the first stream and the second stream are flowing adjacent to each other in the laminar flow microfluidic channel and are allowed to diffuse into each other over the sensing surface of the laminar flow microfluidic channel so as to create a concentration gradient of one analyte relative to its binding pair;
detecting a presence of analytes by their association with the binding pair on the sensing surface of the laminar flow microfluidic channel; and
preventing association of analytes with the sensing surface in a manner that correlates with the quantity of analytes in the first stream.

2. The method of claim 1 wherein the concentration gradient determines a rate of interaction between analytes or their binding pair with the sensing surface.

3. The method of claim 1 wherein the concentration gradient created is a function of the concentration of the analyte in the sample stream.

4. The method of claim 1 wherein the sensing surface is one of the surfaces that bounds fluid flow within the laminar flow microfluidic channel.

5. The method of claim 1 wherein the sensing surface is gold coated.

6. The method of claim 1 wherein the analytes comprise small molecules, antibody/antigen conjugates, nucleic acids, nucleic acid/protein interactions or other protein/protein interactions, or larger particles.

7. The method of claim 6 wherein the larger particles comprise viruses or bacteria.

8. The method of claim 1 wherein the first stream comprises saliva or some other biological fluid sample.

9. The method of claim 1 wherein the first stream comprises a plurality of analytes, and wherein a plurality of corresponding binding pairs are patterned and immobilized over the sensing surface of the laminar flow microfluidic channel.

10. The method of claim 1 wherein the analyte or the binding pair is labeled.

11. The method of claim 1 comprising adjusting a dynamic range of quantitative analysis by choosing an appropriate binding pair concentration.

12. A microfluidic competitive immunoassay device that measures and records interactions between one or more analytes, the device comprising:
a microfluidic channel through which flows at least two fluidic streams adjacent to one another;
one or more analytes in one of a first stream and the analytes binding partner in a second stream flowing adjacent the first stream in the microfluidic channel;
wherein a concentration gradient of the conjugate within the first stream is developed within the microfluidic channel that varies predictably as a function of position within the microfluidic channel but is substantially stable over time;
one or more conjugate(s) or analyte(s) immobilized on a sensing surface portion of one wall of the microfluidic channel;
an imaging assembly that is configured to detect a binding of the analyte or conjugate to the sensing surface, wherein the imaging assembly may discriminate between analytes binding to the sensing surface and analytes present in the bulk fluid flow; and
a processing assembly that receives data a signal from the imaging assembly, wherein the processing assembly is configured to calculate the concentration of analyte in the first stream that correlates with the detection of binding measured at the sensing surface.

13. The device of claim 12 wherein the reversible association between analytes and conjugates within the microfluidic channel is based on specific molecular recognition.

14. The device of claim 12 wherein the imaging assembly comprises a surface plasmon resonance imaging assembly optically coupled to the sensing surface.

15. The device of claim 12 wherein the detection of binding to the sensing surface results in a array of digital data correlated with the position of binding on the sensing surface.

16. The device of claim 12 wherein the imaging assembly is configured to correlate between a location of binding and a concentration of conjugate at that location.

17. The device of claim 12 wherein the processing assembly comprises a processor that runs a digital data analysis algorithm.

18. A microfluidic competitive immunoassay device for measuring two analytes simultaneously in one stream, the device comprising:
a microfluidic channel that comprises three fluid inlets, each inlet configured to receive a fluid stream so as to flow three fluid streams adjacent to one another in the microfluidic channel;
one surface of said microfluidic channel comprising an optically transparent support coated with gold, thereby forming a gold-coated surface;
wherein the gold-coated surface is patterned with at least one conjugate to the analyte sample(s);
a surface plasmon resonance imaging (SPRI) assembly optically coupled to the gold-coated surface, wherein the SPRI assembly is capable of detecting binding events between the analyte(s) and the patterned conjugates on the gold-coated surface; and
a charge-coupled device (CCD) camera coupled to said SPRI assembly, wherein the CCD camera captures images correlated to an amount of analyte bound to the patterned conjugate(s) on the gold-coated surface.

19. The device of claim 18 wherein the microfluidic channel has a width of about 0.1 mm, a height of about 4 mm, and a length of about 30 mm.

20. The device of claim 18 wherein said three fluid streams comprise:
a first stream comprising phosphate buffered saline (PBS);
a second stream comprising PBS containing equimolar concentrations of anti-cortisol and anti-estriol monoclonal antibodies; and
a third stream comprising PBS containing a 2:1 ratio of estriol to cortisol.

21. The device of claim 20 wherein the third stream contains 100 nM estriol and 50 nM cortisol.

22. The device of claim 20 wherein the gold-coated surface is coated with bovine serum albumin (BSA).

23. The device of claim 20 wherein the gold-coated surface is coated from a point where the fluid inlets converge to 22 mm downstream.

24. The device of claim 20 wherein the gold-coated surface is patterned with BSA-cortisol conjugate, BSA-estriol conjugate, and BSA in stripes about 1 mm wide spanning the channel perpendicular to the fluid flow.

25. The device of claim 18 further comprising pumps that are configured to pump the fluid streams through the microfluidic channel at a volumetric rate of about 75 nL/sec.

26. The device of claim 18 wherein said gold coating is about 45 nm thick.
